SEQUENCE LISTING (29 sequences)

SEQ ID NO: 1 - DNA, 711 bp, Artificial Sequence
  Synthetic Bacillus bombysepticus SF3
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttcttctgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 2 - protein, 236 aa, Artificial Sequence
  Synthetic Bacillus bombysepticus SF3
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSSDMRGGEDYGIDTCWYNPSLKENRTDVKPSYEVESLLQILEIVEVTKEKVASF

SEQ ID NO: 3 - DNA, 37 bp, Artificial Sequence
  Synthetic SF0757 gene amplification primer
aagaaggagatataccatgaaatacaaatttatatta

SEQ ID NO: 4 - DNA, 39 bp, Artificial Sequence
  Synthetic SF0757 gene amplification primer
ggtggtggtggtgctcgattaaaatgaagctactttttc

SEQ ID NO: 5 - DNA, 39 bp, Artificial Sequence
  Synthetic primer
gttggagattcgctttctcatgatatgagaggcggagaa

SEQ ID NO: 6 - DNA, 39 bp, Artificial Sequence
  Synthetic primer
ttctccgcctctcatatcatgagaaagcgaatctccaac

SEQ ID NO: 7 - protein, 236 aa, Artificial Sequence
  Synthetic SF0757 S184H mutant protein
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSHDMRGGEDYGIDTCWYNPSLKENRTDVKPSYEVESLLQILEIVEVTKEKVASF

SEQ ID NO: 8 - DNA, 711 bp, Artificial Sequence
  Synthetic SF0757 S184H mutant gene
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcatgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 9 - protein, 236 aa, Artificial Sequence
  Synthetic SF0757 S184H and N206M double mutant protein gene
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSHDMRGGEDYGIDTCWYNPSLKEMRTDVKPSYEVESLLQILEIVEVTKEKVASF

SEQ ID NO: 10 - DNA, 711 bp, Artificial Sequence
  Synthetic SF0757 S184H and N206M double mutant protein gene
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcatgatatgagaggcggagaagattacggcattgatacgtgttggtataatccgagtttgaaagaaatgaggacagatgttaagccgtcttatgaagtggagagtctgctacaaattttagaaattgtagaagtgactaaagaaaaagtagcttcattttaa

SEQ ID NO: 11 - protein, 236 aa, Artificial Sequence
  Synthetic SF0757 S184H and N206M and T208Q triple mutant protein 1
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSHDMRGGEDYGIDTCWYNPSLKEMRQDVKPSYEVESLLQILEIVEVTKEKVASF

SEQ ID NO: 12 - DNA, 711 bp, Artificial Sequence
  Synthetic SF0757 S184H and N206M and T208Q triple mutant protein 1 gene
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcatgatatgagaggcggagaagattacggcattgatacgtgttggtataatccgagtttgaaagaaatgaggcaggatgttaagccgtcttatgaagtggagagtctgctacaaattttagaaattgtagaagtgactaaagaaaaagtagcttcattttaa

SEQ ID NO: 13 - protein, 236 aa, Artificial Sequence
  Synthetic S184H and N206M and V210DQ triple mutant protein 2
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSHDMRGGEDYGIDTCWYNPSLKEMRTDDKPSYEVESLLQILEIVEVTKEKVASF

SEQ ID NO: 14 - DNA, 711 bp, Artificial Sequence
  Synthetic S184H and N206M and V210DQ triple mutant protein 2 gene
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcatgatatgagaggcggagaagattacggcattgatacgtgttggtataatccgagtttgaaagaaatgaggacagatgataagccgtcttatgaagtggagagtctgctacaaattttagaaattgtagaagtgactaaagaaaaagtagcttcattttaa

SEQ ID NO: 15 - DNA, 39 bp, Artificial Sequence
  Synthetic primer
aatccgagtttgaaagaaatgaggacagatgttaagccg

SEQ ID NO: 16 - DNA, 39 bp, Artificial Sequence
  Synthetic primer
cggcttaacatctgtcctcatttctttcaaactcggatt

SEQ ID NO: 17 - DNA, 39 bp, Artificial Sequence
  Synthetic primer
agtttgaaagaaatgaggcaggatgttaagccgtcttat

SEQ ID NO: 18 - DNA, 39 bp, Artificial Sequence
  Synthetic primer
ataagacggcttaacatcctgcctcatttctttcaaact

SEQ ID NO: 19 - DNA, 39 bp, Artificial Sequence
  Synthetic primer
aaagaaatgaggacagatgataagccgtcttatgaagtg

SEQ ID NO: 20 - DNA, 39 bp, Artificial Sequence
  Synthetic primer
cacttcataagacggcttatcatctgtcctcatttcttt

SEQ ID NO: 21 - protein, 40 aa, Artificial Sequence
  Synthetic BANF domain
MSNNNYSNGLNPDESLSASAFDPNLVGPTLPPIPPFTLPT

SEQ ID NO: 22 - DNA, 120 bp, Artificial Sequence
  Synthetic BANF domain gene
atgagtaacaataattatagtaatggcttaaatccggacgaaagtttatctgcatcagcattcgacccgaatcttgtgggtccgaccttgccgcctattccaccatttaccctgccaacc

SEQ ID NO: 23 - DNA, 37 bp, Artificial Sequence
  Synthetic primer
cacacaggaaacagaccatgagtaacaataattatag

SEQ ID NO: 24 - DNA, 27 bp, Artificial Sequence
  Synthetic primer
atataaatttgtatttcatggttggca

SEQ ID NO: 25 - DNA, 39 bp, Artificial Sequence
  Synthetic primer
caccatttaccctgccaaccatgaaatacaaatttatat

SEQ ID NO: 26 - DNA, 39 bp, Artificial Sequence
  Synthetic primer
cctgcaggtcgactctagttaaaatgaagctactttttc

SEQ ID NO: 27 - protein, 40 aa, Artificial Sequence; identical to SEQ ID NO: 21
  Synthetic NTD domain anchor motif

SEQ ID NO: 28 - protein, 115 aa, Artificial Sequence
  Synthetic BclA NTD-CLR domain anchor motif
MSNNNYSNGLNPDESLSASAFDPNLVGPTLPPIPPFTLPTGPTGPTGPTGPTGPTGPTGPTGPTGPTGPTGPTGPTGPTGPTGDTGTTGPTGPTGPTGPTGPTGATGLTGPTGPT

SEQ ID NO: 29 - protein, 253 aa, Artificial Sequence
  Synthetic BclA
MSNNNYSNGLNPDESLSASAFDPNLVGPTLPPIPPFTLPTGPTGPTGPTGPTGPTGPTGPTGPTGPTGPTGPTGPTGPTGPTGDTGTTGPTGPTGPTGPTGPTGATGLTGPTGPTGPSGLGLPAGLYAFNSGGISLDLGINDPVPFNTVGSQFGTAISQLDADTFVISETGFYKITVIANTATASVLGGLTIQVNGVPVPGTGSSLISLGAPIVIQAITQITTTPSLVEVIVTGLGLSLALGTSASIIIEKVA

(19) United States
(12) Patent Application Publication
(10) Pub. No.: US 2020/0032228 A1
(43) Pub. Date: Jan. 30, 2020

(54) VARIANT OF HALOACID DEHALOGENASE SUPERFAMILY PROTEIN AND METHOD OF REDUCING CONCENTRATION OF FLUORINE-CONTAINING COMPOUND IN SAMPLE USING THE SAME

(71) Applicant: Samsung Electronics Co., Ltd., Suwon-si (KR)

(72) Inventors: Yukyung Jung, Hwaseong-si (KR); Taeyong Kim, Daejeon (KR); Jinwoo Park, Suwon-si (KR); Joonsong Park, Seoul (KR); Jinsuk Lee, Suwon-si (KR); Changduk Kang, Gwacheon-si (KR); Jongwon Byun, Suwon-si (KR); Seunghoon Song, Suwon-si (KR)

(21) Appl. No.: 16/378,341

(22) Filed: Apr. 8, 2019

(30) Foreign Application Priority Data
Jul. 24, 2018 (KR) 10-2018-0086025

(51) Int. Cl. C12N 9/14; C12N 15/52; C12N 15/70; C12N 15/74; C07K 14/195

(52) U.S. Cl. CPC C12N 9/14; C12N 15/52; C12Y 308/01002; C12N 15/74; C07K 14/195; C12N 15/70

(57) ABSTRACT

Provided are a variant of a haloacid dehalogenase superfamily protein, a polynucleotide encoding the variant, a recombinant microorganism including the variant, and a method of reducing a concentration of a fluorine-containing compound in a sample using the variant.

### CROSS-REFERENCE TO RELATED APPLICATION

This application claims the benefit of Korean Patent Application No. 10-2018-0086025, filed on Jul. 24, 2018, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 23,158 Byte ASCII (Text) file named “741574_ST25.TXT,” created on Apr. 8, 2019.

### BACKGROUND

### 1. Field

The present disclosure relates to a recombinant microorganism which includes a foreign gene encoding a variant of a haloacid dehalogenase superfamily protein, a composition including the variant for use in removing a fluorine-containing compound in a sample, and a method of reducing a concentration of a fluorine-containing compound in a sample using the variant.

### 2. Description of the Related Art

The emission of greenhouse gases, which have accelerated global warming, is a serious environmental problem, and regulations to reduce and prevent the emissions of greenhouse gases have been tightened. Among the greenhouse gases, fluorinated gases (F-gases), such as perfluorocarbons (PFCs), hydrofluorocarbons (HFCs), and sulfur hexafluoride (SF₆), show low absolute emissions, but have a long half-life and a very high global warming potential, resulting in significantly adverse environmental impacts. The amount of F-gases emitted from the semiconductor and electronics industries, which are major causes of F-gas emissions, has exceeded the assigned amount of the emissions of greenhouse gases and continues to increase. Therefore, costs required for decomposition of greenhouse gases and greenhouse gas emission allowances are increasing every year.

A pyrolysis or catalytic thermal oxidation process has been generally used in the decomposition of F-gases. However, these processes have disadvantages including a limited decomposition rate, emission of secondary pollutants, and high cost. Biological decomposition of F-gases using a microbial biocatalyst has been proposed to overcome the limitations of the existing chemical decomposition process, and also to treat F-gases in a more economical and environmentally-friendly manner.

The haloacid dehalogenase (HAD) superfamily is a superfamily of enzymes that include phosphatases, phosphonatases, P-type ATPases, beta-phosphoglucomutases, phosphomannomutases, and dehalogenases. HAD enzymes are involved in a variety of cellular processes ranging from amino acid biosynthesis to detoxification.

There is a need for a variant of the HAD superfamily for use in the biological decomposition of F-gases. Such variants are provided herein.

### SUMMARY

Provided herein is a variant of a haloacid dehalogenase (HAD) superfamily protein. In an aspect of the disclosure, the variant haloacid dehalogenase superfamily (HAD) protein comprises the substitution of at least one amino acid residue corresponding to positions N206, T208, or V210 of SEQ ID NO: 2, alone or together with the substitution of an amino acid residue corresponding to position S184 of SEQ ID NO: 2. In additional aspects, the substitution of the amino acid residue corresponding to position S184 of SEQ ID NO: 2 is S184H, S184K, or S184R; the substitution of the amino acid residue corresponding to position N206 of SEQ ID NO: 2 is N206M, N206G, N206A, N206V, N206L, N206I, N206F, N206W, or N206P; the substitution of the amino acid residue corresponding to position T208 of SEQ ID NO: 2 is T208Q, T208S, T208C, T208Y, or T208N; and the substitution of the amino acid residue corresponding to position V210 of SEQ ID NO: 2 is V210D or V210E.

Also provided is a composition including the variant, and a recombinant microorganism comprising the variant or a polynucleotide encoding the variant, optionally in a vector, which are useful for reducing the amount or concentration of a fluorine-containing compound in a sample.

Further provided is a polynucleotide encoding the variant and vector comprising the polynucleotide.

A method of reducing the concentration of a fluorine-containing compound in a sample also is provided, the method including contacting a sample including a fluorine-containing compound with the variant HAD protein or recombinant microorganism described herein to reduce the concentration of the fluorine-containing compound in the sample.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a pET-SF0757 vector;

FIG. 2 is a vector map of a pTrc-BANF-SF0757 vector;

FIG. 3 is a schematic diagram of a glass Dimroth reflux condenser; and

FIG. 4 shows a diagram of a microbubble process.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. Expressions such as “at least one of,” when preceding a list of elements, modify the entire list of elements and do not modify the individual elements of the list.

Additional aspects will be set forth in part in the description which follows and, in part, will be apparent from the description, or may be learned by practice of the presented embodiments.

The terms “gene” or “polynucleotide” as used herein may refer to a fragment of a nucleic acid that expresses a particular protein. A gene may include regulatory sequences of a coding region or regulatory sequences of a non-coding region including a 5′-non coding sequence and a 3′-non coding sequence. The regulatory sequences may include a promoter, an enhancer, an operator, a ribosome-binding site, a polyA-binding site, a terminator region, etc.

The term “sequence identity” of a nucleic acid or a polypeptide as used herein refers to a degree of identity between bases or amino acid residues of sequences after being aligned to best match in a certain comparative region. The sequence identity is a value measured by comparing two sequences in a certain comparative region through optimal alignment of the two sequences, wherein some portions of the sequences in the comparative region may be added or deleted compared to a reference sequence. A percentage of sequence identity may be, for example, calculated as follows: two sequences that are optimally aligned are compared in the entire comparative region; the number of locations where the same amino acids or nucleic acids appear in both sequences is determined to be the number of matching locations; the number of matching locations is divided by the total number of locations (i.e., the size of a range) in the comparative region; and the result of the division is multiplied by 100 to obtain the percentage of the sequence identity. The percentage of the sequence identity may be determined using a known sequence comparison program, such as BLASTN or BLASTP (NCBI), CLC Main Workbench (CLC bio), or MegAlign™ (DNASTAR Inc). Unless otherwise mentioned in the specification, the selection of the parameters used to execute the program may be as follows: E-value=0.00001 and H-value=0.001.

Different levels of sequence identity may be used to identify peptides or polynucleotides having the same or similar functions or activities of different species. For example, the sequence identity may be 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, 99% or greater, or 100%.

An aspect of the present disclosure provides a variant of a haloacid dehalogenase superfamily (HAD) protein, the variant including amino acid alteration in an amino acid residue corresponding to position S184 of SEQ ID NO: 2 and at least one amino acid residue corresponding to positions N206, T208, and V210 of SEQ ID NO: 2; or amino acid alternation in at least one amino acid residue corresponding to positions N206, T208, and V210 of SEQ ID NO: 2.

According to certain embodiments, the amino acid alteration in the position S184 may include substitution for S184H, S184K, or S184R. The amino acid alteration in the position N206 may include substitution for N206M, N206G, N206A, N206V, N206L, N206I, N206F, N206W, or N206P. The amino acid alteration in the position T208 may include substitution for T208Q, T208S, T208C, T208Y, or T208N. The amino acid alteration in the position V210 may include substitution for V210D or V210E.

According to certain embodiments, the variant may have amino acid alterations in the amino acid residues corresponding to positions S184 and N206 of SEQ ID NO: 2; alterations in the amino acid residues corresponding to positions S184, N206, and T208 of SEQ ID NO: 2; or alterations in the amino acid residues corresponding to positions S184, N206, and V210 of SEQ ID NO: 2.

Regarding the variant, the amino acid alteration may include substitution for S184H and N206M in an amino acid sequence of SEQ ID NO: 2; substitution for S184H, N206M, and T208D in an amino acid sequence of SEQ ID NO: 2; or substitution for S184H, N206M, and V210D in an amino acid sequence of SEQ ID NO: 2.

The HAD superfamily protein may be a phosphatase, phosphonatase, P-type ATPase, beta-phosphoglucomutase, phosphomannomutase, or a dehalogenase. The HAD superfamily protein may include an HAD domain. The HAD superfamily protein may be a phospholipid-translocating ATPase belonging to EC 3.6.3.1, 3-deoxy-D-manno-octulosonate (KDO) 8-phosphate phosphatase belonging to EC 3.1.3.45, mannosyl-3-phosphoglycerate phosphatase belonging to EC 3.1.3.70, phosphoglycolate phosphatase belonging to EC 3.1.3.18, or HAD belonging to EC 3.8.1.2.

The ATPase may be a putative lipid-flipping enzyme involved in cold tolerance in Arabidopsis. The KDO 8-phosphate phosphatase may catalyze the final step in the biosynthesis of KDO, which is a component of lipopolysaccharide in Gram-negative bacteria. The mannosyl-3-phosphoglycerate phosphatase may hydrolyse mannosyl-3-phosphoglycerate to form osmolyte mannosylglycerate. The phosphoglycolate phosphatase may catalyze the dephosphorylation of 2-phosphoglycolate.

The HAD superfamily protein or a variant thereof may have a sequence identity of 85% or greater, 90% or greater, 95% or greater, 96% or greater, 97% or greater, 98% or greater, or 99% or greater with respect to an amino acid sequence of SEQ ID NO: 2, 7, 9, 11, or 13.

In an embodiment, the variant may have an activity of an enzyme belonging to the HAD superfamily protein, and for example, may have an activity of a haloacid dehalogenase belonging to EC 3.8.1.2.

The alteration may include substitution of the indicated position with an amino acid that is modified after translation. The alteration may include substitution of the indicated position with one of 19 amino acids other than the corresponding amino acid among 20 natural amino acids. Amino acids used herein and abbreviations thereof are shown in Table 1.

TABLE 1

| | |
|---|---|
| Abbreviation | Amino acid |

| | | |
|---|---|---|
| A | Ala | Alanine |
| C | Cys | Cysteine |
| D | Asp | Aspartic acid |
| E | Glu | Glutamic acid |
| F | Phe | Phenylalamine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Regarding the variant, each amino acid substituted at each of positions 184, 206, 208, and 210 of SEQ ID NO: 2 may be in a relationship of “conservative substitution” to each other. The terms “conservative” or “conservative substitution” as used herein refer to substitution of first amino acid with a similar amino acid in terms of the amino acid characteristics. The first amino acid may include H184, K184, or R184; M206, G206, A206, V206, L206, I206, F206, W206, or P206; Q208, S208, C208, Y208, or N208; D210 or E210E in the amino acid sequence of SEQ ID NO: 2. For example, when a non-aliphatic amino acid residue (e.g., Ser) at a specific position is substituted with an aliphatic amino acid residue (e.g., Leu), a substitution with a different aliphatic amino acid (e.g., Ile or Val) at the same position is referred to as a conservative mutation. In addition, the amino acid characteristics include size of the residue, hydrophobicity, polarity, charge, pK-value, and other amino acid characteristics known in the art. Accordingly, a conservative mutation may include substitution, such as basic for basic, acid for acid, polar for polar, and the like. Conservative substitutions may be made, for example, according to Table 2 below which describes a generally accepted grouping of amino acid characteristics.

TABLE 2

| Set | Amino acid |
|---|---|
| Non-polar | G A V L I M F W P |
| Polar | S T C Y N Q |
| Acidic | D E |
| Basic | K R H |

The term “corresponding” as used herein refers to an amino acid position of a protein of interest that aligns with the mentioned position of a reference protein (e.g., position S184 of SEQ ID NO: 2) when amino acid sequences of the protein of interest and the reference protein (e.g., SEQ ID NO: 2) are aligned using an art-acceptable protein alignment program, such as the BLAST pairwise alignment or the well known Lipman-Pearson Protein Alignment program. The protein of interest may be HAD, which belongs to, for example, EC 3.8.1.2. The database (DB) in which the reference sequence is stored may be a Reference Sequence (RefSeq) non-redundant protein database of NCBI. The parameters used for the sequence alignment may be as follows: E-value 0.00001 and H-value 0.001.

A protein obtained according to the alignment conditions above and having an amino acid residue corresponding to position S184 of the amino acid sequence of SEQ ID NO: 2 may be a homolog of the amino acid sequence of SF0757 (SEQ ID NO: 2). The homologs may have 85% or more sequence identity to the amino acid sequence of SEQ ID NO: 2.

In one embodiment, the variant HAD protein comprises SEQ ID NO: 2 with one or more of the following substitutions:
(a) N206M, N206G, N206A, N206V, N206L, N206I, N206F, N206W, or N206P;
(b) T208Q, T2085, T208C, T208Y, or T208N; and/or
(c) V210D or V210E;
   and optionally further comprises a substitution selected from S184H, S184K, or S184R.

Another aspect of the present invention provides a polynucleotide encoding the variant of the HAD superfamily protein.

The polynucleotide may be linked to a polynucleotide encoding an anchoring motif that causes a variant polypeptide to be expressed on a cell surface. The fusion may be accomplished by linking between a C-terminus of the anchoring motif and an N-terminus of the variant polypeptide.

The anchoring motif may include a transmembrane portion and a linker portion disposed in a direction away from the cell surface from the transmembrane portion. The anchoring motif may be selected from a membrane protein, a lipoprotein, and an autotransporter. The anchoring motif may be BclA of Bacillus, OmpA, Lpp-OmpA, OmpC, OmpS, LamB, OmpC, Lpp-OmpC, PhoE, and FadL of Eschrichia, OmpC of Salmonella, OprF of Pseudomonas, and AIDA-I of pathogenic Escherichia, or a fragment thereof.

The anchoring motif may be BclA having an amino acid sequence of SEQ ID NO: 27, SEQ ID NO: 28, or SEQ ID NO: 29.

Another aspect of the present invention provides a vector including the polynucleotide encoding the variant. For use as a vector, any vehicle known in the art that may be used to introduce the polynucleotide to a microorganism may be used. The vector may be, for example, a plasm id or a viral vector.

Another aspect of the present disclosure provides a recombinant microorganism including the HAD variant protein described herein or polynucleotide encoding the variant. The polynucleotide may optional be part of a vector. The polynucleotide may be present in the microorganism chromosomally or extra-chromosomally (e.g., the polynucleotide may integrate into the host cell chromosome or may remain in the host cell without integrating into the host cell chromosome). The microorganism may express the polynucleotide to produce a variant protein described herein. The variant protein may then be present inside the cell, expressed on the cell surface, or released outside the cell.

The recombinant microorganism may be bacteria or fungi, and the bacteria may be Gram-positive or Gram-negative. The Gram-negative bacteria may belong to the Enterobacteriaceae family. The Gram-negative bacteria may belong to the genus Escherichia, the genus Samonella, the genus Xanthobacter, or the genus Pseudomonas. The microorganism belonging to the genus Escherichia may be E. coli. The microorganism belonging to the genus Pseudomonas may be P. saitens SF1. The microorganism belonging to the genus Xanthobacter may be X. autotrophicus. The Gram-positive bacteria may belong to the genus Corynebacterium or the genus Bacillus. The microorganism belonging to the genus Bacillus may be B. bombysepticus SF3.

Another aspect of the present disclosure provides a composition for use in reducing a fluorine-containing compound in a sample, the composition including the recombinant microorganism that includes the variant of the HAD superfamily protein or the polynucleotide encoding the variant. The fluorine-containing compound referred to herein may be an alkane compound having 1 to 12 carbon atoms substituted with at least one fluorine. The term “fluorine-containing compound” as used herein may be represented by Formula 1 or 2:

C(R₁)(R₂)(R₃)(R₄) <Formula 1>

(R₅)(R₆)(R₇)C—[C(R₁₁)(R₁₂)]n-C(R₈)(R₉)(R₁₀). <Formula 2>

In Formula 1, R₁, R₂, R₃, and R₄ may each independently be fluorine (F), chlorine (Cl), bromine (Br), iodine (I), or hydrogen (H), wherein at least one selected from R₁, R₂, R₃, R₄ is F.

In Formula 2, n may be an integer from 0 to 10, and when n is equal to or greater than 2, each of R₁₁ is identical to or different from each other and each of R₁₂ is identical to or different from each other, and R₅, R₆, R₇, R₈, R₉, R₁₀, R₁₁, and R₁₂ may each be independently F, Cl, Br, I, or H, wherein at least one selected from R₅, R₆, R₇, R₈, R₉, R₁₀, R₁₁, and R₁₂ is F.

In some embodiments, the fluorine-containing compound may be, for example, CHF3, CH2F2, CH3F, or CF4. The term “removal” as used herein refers to any reduction in the concentration of the fluorine-containing compound. The reduction includes partial or complete reduction, i.e., a concentration at or near zero.

The composition may include a variant of the HAD superfamily protein witih activity belonging to EC 3.8.1.2. The composition may include the recombinant microorganism, a lysate thereof, or a water soluble material fraction of the lysate.

The removal of the fluorine-containing compound may include any reduction in the concentration of the fluorine-containing compound in the sample, such as cleavage of a C—F bond of the fluorine-containing compound, conversion of the fluorine-containing compound into a different material, or accumulation of the fluorine-containing compound in a cell. The conversion of the fluorine-containing compound may include introduction of a hydrophilic group, such as a hydroxyl group, to the fluorine-containing compound, or introduction of a carbon-carbon double bond or a carbon-carbon triple bond to the fluorine-containing compound.

The sample may be a liquid sample, a gaseous sample, or a combination of the two. For example, the sample may be sewage, waste gas, or both.

Another aspect of the present invention provides a method of reducing a concentration of a fluorine-containing compound in a sample, the method includingcontacting the variant HAD protein described herein, or recombinant microorganism including the variant HAD protein or polynucleotide encoding the variant HAD protein, with the sample including the fluorine-containing compound represented by Formula 1 or 2 to reduce the concentration of the fluorine-containing compound in the sample:

C(R₁)(R₂)(R₃)(R₄) <Formula 1>

(R₅)(R₆)(R₇)C—[C(R₁₁)(R₁₂)]n-C(R₈)(R₉)(R₁₀) <Formula 2>

In Formula 1, R₁, R₂, R₃, and R₄ may each independently be fluorine (F), chlorine (Cl), bromine (Br), iodine (I), or hydrogen (H), wherein at least one selected from R₁, R₂, R₃, R₄ is F.

In Formula 2, n may be an integer from 0 to 10, and when n is equal to or greater than 2, each of R₁₁ is identical to or different from each other and each of R₁₂ is identical to or different from each other, and R₅, R₆, R₇, R₈, R₉, R₁₀, R₁₁, and R₁₂ may each be independently F, Cl, Br, I, or H, wherein at least one selected from R₅, R₆, R₇, R₈, R₉, R₁₀, R₁₁, and R₁₂ is F.

The recombinant microorganism may further include a second polynucleotide encoding a dehalogenase. In some embodiments, the recorminant microorganism contains a first dehalogenase that is a variant HAD protein as described herein linked to an anchoring motif (or contains a polynucleotide encoding same), and contains a second dehalogenase or polynucleotide encoding same that is not linked to an anchoring motif (or polynucleotide encoding same), which can be the same or different from the first dehalogenase. Or, vice versa, the first dehalogenase that is a variant HAD protein can be free of an anchoring moiety and the second dehalogenase can include an anchoring motif. Accordingly, a dehalogenase may be simultaneously expressed on the cell surface and inside the cell. The second dehalogenase (or polynucleotide encoding same) may be endogenous or exogenous, recombinant microorganism may allow the dehalogenase to be expressed in the cell intrinsically or by recombination.

The recombinant microorganism may have activity of reducing the concentration of the “fluorine-containing compound” in the sample. The fluorine-containing compound may be CH₃F, CH₂F₂, CHF₃, CF₄, CH₂FCOOH, or a mixture thereof.

The removal of the fluorine-containing compound may include any reduction in the concentration of the fluorine-containing compound in the sample, such as cleavage of a C—F bond of the fluorine-containing compound, conversion of the fluorine-containing compound into a different material, or accumulation of the fluorine-containing compound in a cell.

The sample may be a liquid sample, a gaseous sample, or a combination thereof. For example, the sample may be sewage or waste gas. For example, the sample may be a sludge. The term “sludge” as used herein refers to a semi-solid slurry, and may be obtained, for example, as sewage sludge from wastewater treatment processes, or as a settled suspension obtained from conventional drinking water treatment or numerous other industrial processes.

Contacting the recombinant microorganism with the sample may be performed in any suitable manner, such as in a liquid phase or a gaseous phase. The contacting may include culturing the recombinant microorganism in the presence of the fluorine-containing compound. The contacting may be performed in a closed container, for example, a sealed, air-tight container. The contacting may be performed when the growth state of the recombinant microorganism is in an exponential phase or a stationary phase. The culturing may be performed under aerobic or anaerobic conditions. The contacting may be performed under conditions where the recombinant microorganism may survive in a closed container, for example, an air-tight container. Such conditions for the survival of the recombinant microorganism may include conditions where the recombinant microorganism may proliferate or may be allowed to be in a resting state.

The contacting may include passive contacting and active contacting. The term ‘passive contacting’ refers to a contacting without an external driving force and the term ‘active contacting’ refers to a contacting with an external driving force. Active contacting may be achieved in a way that the fluorine-containing compound is injected in the form of bubbles into a solution containing the recombinant microorganism and/or the recombinant microorganism is sprayed. For example, the contacting may be achieved by blowing the sample into a medium or a culture broth. For the injection of the sample, the sample may be blown from the bottom of the medium or the culture broth to the top thereof. The injection of the sample may be achieved by making droplets of the sample. The contacting may be performed in a batch or continuous manner. The contacting may be performed repeatedly, such as two or more times, for example, three times, five times, or ten times or more. The contacting may be continued or repeated until the fluorine-containing compound is reduced to a desired concentration.

The recombinant microorganism may be in the form of a thin film layer. Such a thin film layer of the recombinant microorganism may be a liquid thin film layer. The fluorine-containing compound may be in the form of a gaseous thin film layer. The liquid thin film layer of the recombinant microorganism and the gaseous thin film layer of the fluorine-containing compound may contact each other.

The recombinant microorganism may be subjected to a circulation process, and in this regard, the contact area or the contact time of the recombinant microorganism with the fluorine-containing compound may increase. Such a circulation process may increase a mass transfer coefficient (KLa) value, as well as the amount (or rate) of decomposition of the fluorine-containing compound.

Regarding the method, the contacting may further be performed using an exhaust gas decomposition device including one or more reactors each including one or more first inlets and one or more first outlets, wherein the sample is injected into such an exhaust gas decomposition device, and

wherein the recombinant microorganism is injected into the exhaust decomposition device through the one or more first inlets, so that the recombinant microorganism may contact the sample and the resulting mixture may be discharged through the one or more outlets.

In this regard, the exhaust gas decomposition device may include a second inlet and a second outlet, and the sample may be injected through the second inlet and discharged through the second outlet. In such a configuration, the recombinant microorganism may move in a direction opposite to a direction in which the sample moves. A fluid thin film including the recombinant microorganism may be formed on an inner wall of the one or more reactors.

Regarding the method, the exhaust gas decomposition device may further include a first circulation line for re-supplying at least a portion of a fluid to the one or more first inlets, wherein the fluid contains the recombinant microorganism. The sample including the fluorine-containing compound may remain inside the one or more reactors. In addition, the exhaust gas decomposition device may further include a second inlet and a second outlet, wherein the sample may be supplied into the one or more reactors through the second inlet, discharged to the outside of the one or more reactors through the second outlet, and move in a second direction within the one or more reactors. The second direction may be different from, for example, opposite to, the direction in which the fluid containing the recombinant microorganism moves. In addition, in at least one of a fluid collection zone at the inner bottom of the one or more reactors and a fluid reaction zone at the inner top of the one or more reactors of the exhaust gas decomposition device, the fluid including the recombinant microorganism and the sample including the fluorine-containing compound may contact each other, thereby decomposing the fluorine-containing compound. In the exhaust gas decomposition device, a fluid thin film including the fluid containing the recombinant microorganism may contact a fluid including the sample.

Regarding the method, the exhaust gas decomposition device may further include a structure inside the one or more reactors, wherein the structure may be configured to increase a contact area between the fluid including the recombinant microorganism and the sample including the fluorine-containing compound. For example, the structure may include at least one selected from a packing material and a reflux tube, but the embodiments of the present disclosure are not limited thereto. Any structure configured to increase a contact area between the fluid including the recombinant microorganism and the sample including the fluorine-containing compound may be included. The ‘packing material’ may be inert solid material. The packing material may have various shapes. The packing material may be the same material used in the packing of a packed bed tower. The packing material may be made of plastic, magnetic material, steel, or aluminum. The packing material may have very thin thickness. The packing material may have a ring shape such as rashing ring, pall ring, and berl saddle, a saddle type, and protrusion type. The packing material may be irregularly packed in the packed bed reactor. The packing material may efficiently increase contact between the fluorine-containing compound with a microorganism present in a liquid. The time or opportunity to contact between the fluorine-containing compound with a microorganism can be maximized by forming a thin film of microorganisms on the surface of the packing material as well as on the inner surface of the reactor. In addition, the one or more first inlets may be connected to the fluid reaction zone at the inner top of the one or more reactors in the exhaust gas decomposition device, to thereby supply the fluid including the recombinant microorganism through the one or more first inlets.

Regarding the method, the fluid including the recombinant microorganism may be collected in the fluid collection zone at the inner bottom of the one or more reactors in the exhaust gas decomposition device. The sample including the fluorine-containing compound supplied into the one or more reactors through the second inlet may pass through, in the form of bubbles, the collected fluid including the recombinant microorganism to be transferred to the fluid reaction zone at the inner top of the one or more reactors, and then, may be discharged to the outside of the one or more reactors through the second outlet.

Regarding the method, an aspect ratio (H/D) of the height H to the diameter D of the one or more reactors in the exhaust gas decomposition device may be 2 or greater, 5 or greater, 10 or greater, 15 or greater, 20 or greater, or 50 or greater.

Regarding the method, the exhaust gas decomposition device may be arranged in a manner such that the side-wall of the one or more reactors, or some other internal surface thereof, may be tilted or inclined at an angle less than 90° or greater than 90°. For example, the side-wall or other internal surface thereof can be tilted or inclined in a range of about 30° to about 150° (e.g., about 30° to less than 90° or greater than 90° to about 150°), about 70° to about 110° (e.g., about 70° to less than 90° or greater than 90° to about 110°), about 80° to about 100° (e.g., about 80° to less than 90° or greater than 90° to about 100°), or about 50° to about 90° (e.g., about 50° to less than 90°), with respect to the surface of the earth.

Regarding the method, the one or more reactors in the exhaust gas decomposition device may rotate. The fluid including the recombinant microorganism may be a liquid, and the sample including the fluorine-containing compound may be a gas.

The variant according to any of the above-described embodiments and the polynucleotide encoding the variant may be used to remove the fluorine-containing compound in the sample or to produce the variant.

The recombinant microorganism according to any of the above-described embodiments may be used to remove the fluorine-containing compound in the sample.

The composition including the HAD superfamily protein according to any of the above-described embodiments may be used to remove the fluorine-containing compound in the sample.

The method of reducing the concentration of the fluorine-containing compound in the sample according to any of the above-described embodiments may efficiently remove the fluorine-containing compound from the sample.

### EXAMPLE 1

### Preparation of a Recombinant Microorganism that Includes a SF0757 Gene

A gene SF0757 (SEQ ID NO: 1), which encodes a dehalogenase, was amplified from Bacillus bombysepticus SF3, a strain deposited at the Korean Collection for Type Culture (KCTC), which is an international depository authority under the Budapest Treaty as Accession No. KCTC 13220BP, on Feb. 24, 2017.

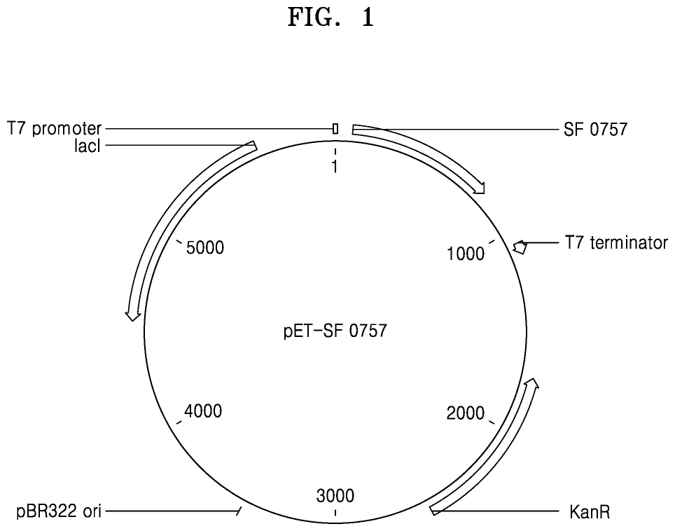

Briefly, B. bombysepticus SF3 was cultured overnight in an LB medium while being stirred at a temperature of 30° C. at a speed of 230 rpm, and genomic DNA thereof was isolated using a total DNA extraction kit (Invitrogen Biotechnology). PCR was performed using the genomic DNA as a template and a set of primers having nucleotide sequences of SEQ ID NO: 3 and SEQ ID NO: 4, so as to amplify and obtain SF0757 genes. The genes thus amplified were each independently ligated with a pET28a vector (Novagen, Cat. No. 69864-3) which was cut by restriction enzymes, such as NcoI and XhoI, by using an InFusion Cloning Kit (Clontech Laboratories, Inc.), so as to prepare a pET-SF0757 vector. FIG. 1 is a vector map of the pET-SF0757 vector. Here, a SF0757 protein has an amino acid sequence of SEQ ID NO: 2.

Next, the prepared pET-SF0757 vector was introduced to E. coli BL21 by a heat shock method, and then, cultured in an LB plate agar containing 50 μg/mL of kanamycin. Strains showing kanamycin resistance were selected from the plate, and a strain finally selected from therefrom was designated as recombinant E. coli BL21/pET-SF0757.

### EXAMPLE 2

### Preparation of Recombinant E. Coli which Expresses a Variant Gene of the SF0757

A variant was prepared to improve the activity of the SF0757 protein on the removal of a fluorine-containing compound in a sample. Serine at position 184 (hereinafter, referred to as “S184) of the amino acid sequence of SEQ ID NO: 2 was substituted with one of other 19 natural amino acids. Here, each substitution is represented by “S184X” (wherein X indicates each of 19 natural amino acids other than serine). Recombinant E. coli were prepared by introducing a polynucleotide encoding each prepared variant. The effect of these recombinant E. coli strains on the removal of CF4 in a sample was then confirmed.

The S184H variant (referred to herein as the “SM” variant) was selected for further experiments. The SM variant was created using primer sets of SEQ ID NOs: 5 and 6 to introduce the S184H into the SF0757 protein. The SM variant had an amino acid sequence of SEQ ID NO: 7, and a nucleotide sequence of SEQ ID NO: 8.

A variant having both S184H and N206M substitutions (hereinafter, referred to as “DM”) was prepared by introducing N206M substitution into the SM variant. Additional variants also were produced with substitutions at positions 208 or 210. In particular, a variant having S184H, N206M, and T208Q substitutions (hereinafter, referred to as “TM1”) was prepared by introducing S208Q substitution to a variant having S184H and N206M, and a variant having S184H, N206M, and V210D substitutions (hereinafter, referred to as “TM2”) was prepared by introducing V210D substitution to a variant having S184H and N206M.

In the preparation of the variants DM, TM1, and TM2, primer sets used for the introduction of N206M, T208Q, and V210D substitutions were as follows: primer sets of SEQ ID NOs: 17 and 18 for the introduction of the variant having N206M substitution; primer sets of SEQ ID NOs: 17 and 18 for the introduction of the variant having N208Q substitution; and primer sets of SEQ ID NOs: 19 and 20 for the introduction of the variant having N210D substitution. The amino acid sequence of the variant DM and a nucleotide sequence encoding the variant DM are SEQ ID NOs: 9 and 10, respectively, an amino acid sequence of the variant TM1 and a nucleotide sequence encoding the variant TM1 are SEQ ID NOs: 11 and 12, respectively, and an amino acid sequence of the variant TM2 and a nucleotide sequence encoding the variant TM2 are SEQ ID NOs: 13 and 14, respectively. For ease of reference, a summary of the variants is as follows:

| Variant | Mutations | Amino Acid Seq. |
|---|---|---|
| SM | S184H | SEQ ID NO: 7 |
| DM | S184H, N206M | SEQ ID NO: 9 |
| TM1 | S184H, N206M, T208Q | SEQ ID NO: 11 |
| TM2 | S184H, N206M, V210D | SEQ ID NO: 13 |

A more detailed description of the preparation of the variant polypeptides and E. coli strains expressing same are provided in the following paragraphs

(1) Induction of Mutagenesis

The preparation of the S184X variant of SEQ ID NO: 2 was achieved by using the QuikChange II Site-Directed Mutagenesis Kit (Agilent Technology, USA). Site-directed mutagenesis using the kit was performed by using PfuUlta high-fidelity (HF) DNA polymerase for mutagenic primer-directed replication of two plasmid strands with the highest fidelity. The basic procedure utilizes a super-coiled double-stranded DNA (dsDNA) vector with an insert of interest and two synthetic oligonucleotide primers, both containing the desired mutation. The oligonucleotide primers, each complementary to opposite strands of the vector, were extended during temperature cycling by PfuUltra HF DNA polymerase, without primer displacement. Extension of the oligonucleotide primers generated a mutated plasmid containing staggered nicks. Following temperature cycling, the product was treated with DpnI. The DpnI endonuclease (target sequence: 5′-Gm⁶ATC-3′) was specific for methylated and hemimethylated DNA, and was used to digest the parental DNA template for the selection of mutation-containing synthesized DNA. Afterwards, the nicked vector DNA incorporating the desired mutations was then transformed into XL1-Blue supercompetent cells.

(2) Recombinant E. Coli BL21/pET-SF0757mt to which a Gene of the SF0757 Variant was Introduced

PCR was performed by using the pET-SF0757 vector of section (1) as a template, a primer selected from primer sets for each of the variants of section (1), and a PfuUlta HF DNA polymerase, so as to obtain variant vectors including staggered nicks. These vector products were treated with DpnI to select synthesized DNA including the variants (referred to as SF0757mt, wherein “mt” refers to “mutant”). Afterwards, the nicked vector DNA incorporating a desired variant was transformed into XL1-Blue supercompetent cells, thereby cloning the desired pET-SF0757mt vector.

Lastly, the cloned pET-SF0757mt vector was introduced into a strain of E. coli BL21 in the same manner as in section (1), and a strain selected therefrom was designated as a recombinant E. coli BL21/pET-SF0757mt strain expressing the particular variant protein encoded by the SF0757mt nucleic acid (i.e., the SM, DM, TM1, or TM2 variant).

(3) Recombinant E. Coli W3110/pTrc-BANF-SF0757mt to which a Gene of the SF0757 Variant was Introduced

Recombinant E. coli expressing a fusion protein on a cell surface was prepared, the fusion protein including the N-terminus of the W3100 SF0757 variant and BANF having an amino acid sequence of SEQ ID NO: 21 that are linked therewith. Hereinafter, the resulting strain is referred to as W3110/pTrc-BANF-SF0757mt. The term “BANF” refers toan N-terminus domain of Bacillus anthracis-derived exosporium protein (BclA), such as that comprising the amino acid sequence of SEQ ID NO: 21. A synthetic polynucleotide encoding the BANF (synthesized by Cosmogenetech Inc., Korea) has a nucleotide sequence of SEQ ID NO: 22.

In the presence of the synthesized polynucleotide encoding a BANF polypeptide, PCR was performed using a set of primers having the oligonucleotide sequences of SEQ ID NOs: 23 and 24. BANF-corresponding DNA fragments were thus obtained by amplification. Then, PCR was performed by using the genomic DNA of the B. bombysepticus SF3 as a template and a set of primers having oligonucleotide sequences of SEQ ID NOs: 25 and 26, so as to obtain SF0757-corresponding DNA fragments by amplification. PCR was performed again by using each of the amplified BANF and SF0757 DNA fragments as a template and a set of primers having oligonucleotide sequences of SEQ ID NOs: 23 and 26, so as to obtain a BANF-SF0757 fusion polynucleotide. Using the InFusion Cloning Kit (Clontech Laboratories, Inc.), the BANF-SF0757 fusion polynucleotide was ligated into a pTrc99A vector (Pharmacia Biotech, Uppsala, Sweden) (4.17 kb, bla, trc promoter), which was digested with restriction enzymes, NcoI and XbaI, to create the pTrc-BANF-SF0757 vector.

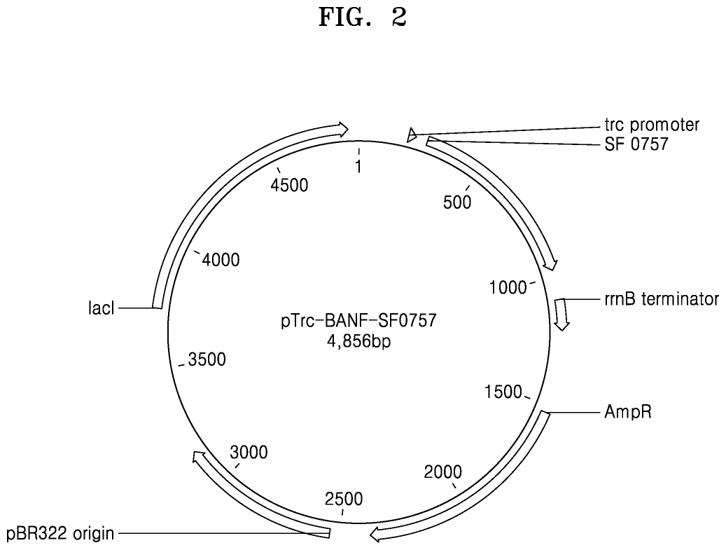

FIG. 2 is a vector map of the pTrc-BANF-SF0757 vector.

Next, PCR was performed by using the prepared pTrc-BANF-SF0757 vector as a template, a primer selected from primer sets for each of the variants of section (1), and a PfuUlta HF DNA polymerase, so as to obtain variant vectors including staggered nicks. These vector products were treated with DpnI to select synthesized DAN including the variants. Afterwards, the nicked vector DNA incorporating a desired variant was transformed into XL1-Blue supercompetent cells, thereby cloning thepTrc-BANF-SF0757mt vector.

Lastly, the cloned pTrc-BANF-SF0757 vector was introduced into a strain of E. coli W3110 in the same manner as in section (1), and a strain finally selected therefrom was designated as a recombinant E. coli W3110/pTrc-BANF-SF0757mt. Again, SF0757mt indicates a nucleic acid encoding the desired variant (i.e., the SM, DM, TM1, or TM2 variants described above).

### EXAMPLE 3

### Effect of Recombinant E. Coli Including the SF0757 Variant Introduced Thereto on the Removal of CF4 in a Sample

In this section, the effect of the E. coli BL21/pET-SF0757mt strains of Example 2 on the removal of CF4 in a sample was confirmed.

(1) Decomposition of Fluorine-Containing Compound by a Circulation Process

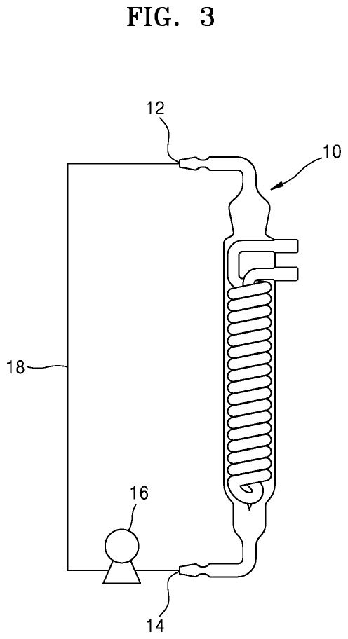

Referring to FIG. 3, 50 ml of an LB medium and 200 ppm of CF4 gas were added to a glass Dimroth coil reflux condenser 10 (a reactor length 350 mm, an exterior diameter 35 mm, and an interior volume: 200 mL) that was sterilized and vertically oriented, and then, the LB medium was subjected to circulation via a circulation pump 16. FIG. 3 provides a schematic illustration of the glass Dimroth reflux condenser 10. The LB medium was supplied to an inlet 12 of an upper portion of the condenser 10, flowed through an inner wall of the condenser 10, and discharged to an outlet 14 of a lower portion of the condenser 10. The discharged LB medium was re-supplied to the inlet 12 along a circulation line 18. To maintain the temperature, an inner screwed pipe [please add a label for this element, which is shown in the drawing but not yet labeled] of the condenser 10 was connected to a constant temperature zone of 30° C. The circulation rate of the LB medium was maintained at 4 mL/min. After 72 hours, the amount of the CF4 gas in the condenser was measured by gas chromatography mass-spectrum (GC-MS). It was confirmed that there was no change in the amount of CF4 gas.

Subsequently, the recombinant microorganisms DM and TM1 of Example 2 and a negative control group were each separately inoculated on an LB medium and circulated in the condenser 10. The E. coli to which an empty vector was introduced was used as a negative control group. The circulation rate of the LB culture medium was about 4 mL/min, and the temperature inside the condenser 10 was maintained at 30° C. After strain inoculation and the elapse of 72 hours, the amount of CF4 gas in the condenser 10 was confirmed by GC-MS. The decomposition rate of CF4 was calculated according to Equation 1:

Decomposition rate of CF4=[(Initial amount of CF4−Amount of CF4 after reaction)/Initial amount of CF4]×100. <Equation 1>

No change in the level of CF4 was observed using the control strain of E. coli with the empty vector. The results using the test strains are shown in Table 3:

TABLE 3

| Strain | Decomposition rate of CF4 (%) |
|---|---|
| DM(S184H N206M) | 10 |
| TM1(S184H N206M T208Q) | 13 |

As shown in Table 3, a significant reduction in the concentration of CF4 was observed after 72 hours of contact with the variant strains as compared with the negative control group (0% reduction).

Table 4 shows the results of 48 hour-culturing and 144 hour-culturing of strains SM and DM of Example 2 according to the same circulation process described above, except that the starting concentration of CF4 gas was 1,000 ppm. Again, E. coli transformed with an empty vector was used as a negative control and no decomposition of CF4 was observed.

TABLE 4

Decomposition rate of CF4 (%)

| Strain | 48 hours | 144 hours |
|---|---|---|
| SM (S184H) | 20.5 | 22.9 |
| DM (S184H N206M) | 25.6 | 31.9 |

As shown in Table 4, the variant after 48 and 144 hours of the culture showed a significant reduction in the concentration of CF4 as compared with the control group (0% CF4 reduction), when applying the gas phase circulation process using the microorganism thereto. In addition, the DM strain showed an increase in the decomposition rate of CF4 by 1.25 times and 1.39 times the decomposition rate of the SM strain at 48 hour-culturing and 114 hour-culturing, respectively.

(2) Decomposition of Fluorine-Containing Compound by Microbubble Process

The tested SM, DM, TM2 strains of E. coli BL21/pET-SF0757mt, as well as a control strain (E. coli transformed with an empty vector), were cultured in an LB medium while being stirred at a speed of 230 rpm at a temperature of 30° C. At an OD₆₀₀ of about 0.5, 0.2 mM of IPTG was added thereto, followed by culturing at a temperature of 20° C. while being stirred at a speed of 230 rpm overnight.

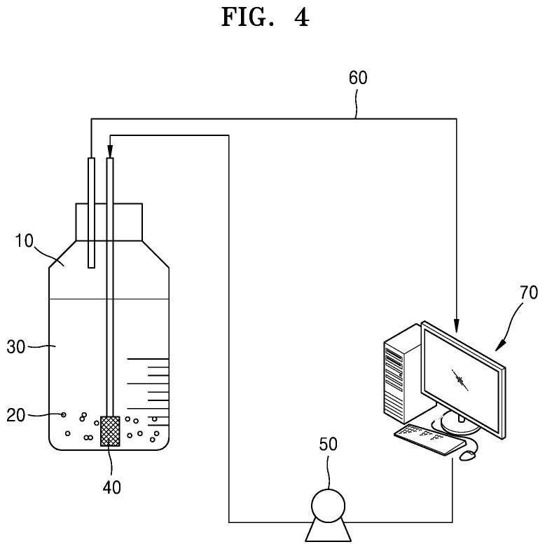

The strain was exposed to a CF4 containing sample using a microbubble process. FIG. 4 illustrates the microbubble process. Referring to FIG. 4, 200 ml of an LB medium 30 and 20 ppm of CF₄ gas 20 were injected into a 500 mL pyrex round media storage bottle-based microbubble reactor 10 that was previously sterilized at a high temperature. A microporous sparger 40 was disposed at the bottom of the microbubble reactor 10 to face the bottom. The microporous sparger 40 had a pore size of 10 um.

The cultured recombinant E. coli was inoculated in an LB medium 30 in the microbubble reactor 10. The initial concentration of the inoculated strain in the LB medium 30 was 5.0, measured on the basis of OD₆₀₀.

CF₄ gas was supplied in the form of bubbles 20 to the LB medium 30 filled in the lower part of the microbubble reactor 10 through the microporous sparger 40 connected to the inlet at the bottom of the microbubble reactor 10, and then, was discharged to the outlet of the upper portion of the microbubble reactor 10. The discharged CF₄ gas was re-supplied to the inlet along a circulation line 60 by a gas pump 50, and the amount of the CF₄ gas was confirmed in real time by an FT-IR gas analyzer 70 connected to the circulation line 60. Here, the circulation speed of the CF₄ gas was 10 volume per volume per minutes (vvm, volume of CF4 circulated by LB medium unit volume per minute). The temperature inside the microbubble reactor 10 was maintained at 30° C., and the pressure inside the microbubble reactor 10 was 1 atm.

The decomposition rate was calculated using Equation 1 below:

CF4 decomposition rate=[(Initial amount of CF4−Amount of CF4 after reaction)/Initial amount of CF4]×100 <Equation 1>

The increase in CF4 reduction rates of the SM, DM, and TM2 strains as compared to the control strain are presented in Table 5.

Compared with wild-type group, the SM, DM, and TM2 strains including the variant protein each showed a significant increase in the activity of reducing CF₄ in the sample.

TABLE 5

| Strain | Decomposition rate of CF4 (%) |
|---|---|
| SM(S184H) | 11.8 |
| DM(S184H N206M) | 12.6 |
| TM2(S184H N206M V210D) | 14.5 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A variant haloacid dehalogenase superfamily (HAD) protein comprising:
substitution of an amino acid residue corresponding to position S184 of SEQ ID NO: 2, and substitution of at least one amino acid residue corresponding to positions N206, T208, and V210 of SEQ ID NO: 2; or
substitution of at least one amino acid residue corresponding to positions N206, T208, or V210 of SEQ ID NO: 2,
wherein the substitution of the amino acid residue corresponding to position S184 of SEQ ID NO: 2 is S184H, S184K, or S184R;
the substitution of the amino acid residue corresponding to position N206 of SEQ ID NO: 2 is N206M, N206G, N206A, N206V, N206L, N206I, N206F, N206W, or N206P;
the substitution of the amino acid residue corresponding to position T208 of SEQ ID NO: 2 is T208Q, T2085, T208C, T208Y, or T208N;
and the substitution of the amino acid residue corresponding to position V210 of SEQ ID NO: 2 is V210D or V210E.

2. The variant protein of claim 1, wherein the variant protein comprises:
substitution of amino acid residues corresponding to positions S184 and N206 of SEQ ID NO: 2;
substitution of amino acid residues corresponding to positions S184, N206, and T208 of SEQ ID NO: 2; or
substitution of amino acid residues corresponding to positions S184, N206, and V210 of SEQ ID NO: 2.

3. The variant of claim 2, wherein the variant protein comprises the following substitutions with reference to SEQ ID NO: 2:
S184H and N206M;
S184H, N206M, and T208D; or
S184H, N206M, and V210D.

4. A polynucleotide encoding the variant haloacid dehalogenase superfamily (HAD) protein of claim 1.

5. The polynucleotide of claim 4, wherein the variant protein comprises:
substitution of amino acid residues corresponding to positions S184 and N206 of SEQ ID NO: 2;
substitution of amino acid residues corresponding to positions S184, N206, and T208 of SEQ ID NO: 2; or
substitution of amino acid residues corresponding to positions S184, N206, and V210 of SEQ ID NO: 2.

6. The polynucleotide of claim 5, wherein the variant protein comprises the following amino acid substitutions:
S184H and N206M;
S184H, N206M, and T208Q; or
S184H, N206M, and V210D.

7. A recombinant vector comprising the polynucleotide of claim 4.

8. The recombinant vector of claim 7, wherein the variant protein comprises:
substitution of amino acid residues corresponding to positions S184 and N206 of SEQ ID NO: 2;
substitution of amino acid residues corresponding to positions S184, N206, and T208 of SEQ ID NO: 2; or
substitution of amino acid residues corresponding to positions S184, N206, and V210 of SEQ ID NO: 2.

9. The recombinant vector of claim 8, wherein the variant protein comprises the following amino acid substitutions:
S184H and N206M;
S184H, N206M, and T208Q; or
S184H, N206M, and V210D.

10. A recombinant microorganism comprising a variant haloacid dehalogenase superfamily (HAD) protein of claim 1 or polynucleotide encoding same.

11. The recombinant microorganism of claim 10, wherein the variant protein comprises:
substitution of amino acid residues corresponding to positions S184 and N206 of SEQ ID NO: 2;
substitution of amino acid residues corresponding to positions S184, N206, and T208 of SEQ ID NO: 2; or
substitution of amino acid residues corresponding to positions S184, N206, and V210 of SEQ ID NO: 2.

12. The recombinant microorganism of claim 11, wherein the variant protein comprises the following amino acid substitutions:
S184H and N206M;
S184H, N206M, and T208Q; or
S184H, N206M, and V210D.

13. The recombinant microorganism of claim 10, wherein the recombinant microorganism belongs to the genus Escherichia, Pseudomonas, or Bacillus.

14. The recombinant microorganism of claim 10, wherein the recombinant microorganism is E. coli, P. saitens SF1, or B. bombysepticus SF3.

15. A method of reducing a concentration of a fluorine-containing compound in a sample, the method comprising:
contacting a sample comprising a fluorine-containing compound represented by Formula 1 or 2 with a variant haloacid dehalogenase superfamily (HAD) protein of claim 1 to reduce the concentration of the fluorine-containing compound in the sample:
C(R₁)(R₂)(R₃)(R₄) <Formula 1>
(R₅)(R₆)(R₇)C—[C(R₁₁)(R₁₂)]n-C(R₈)(R₉)(R₁₀) <Formula 2>
wherein, in Formulae 1 and 2:
n is an integer of 0 to 10,
R₁, R₂, R₃, and R₄ are each independently fluorine (F), chlorine (Cl), bromine (Br), iodine (I), or hydrogen (H), wherein at least one selected from R₁, R₂, R₃, and R₄ is F, and
R₅, R₆, R₇, R₈, R₉, R₁₀, R₁₁, and R₁₂ are each independently F, Cl, Br, I, or H, wherein at least one selected from R₅, R₆, R₇, R₈, R₉, R₁₀, R₁₁, and R₁₂ is F.

16. The method of claim 15, wherein the variant protein is in a microorganism comprising a polynucleotide that encodes the variant protein, and the sample is contacted with the variant protein by contacting the sample with the microorganism.

17. The method of claim 16, wherein the variant protein comprises:
substitution of amino acid residues corresponding to positions S184 and N206 of SEQ ID NO: 2;
substitution of amino acid residues corresponding to positions S184, N206, and T208 of SEQ ID NO: 2; or
substitution of amino acid residues corresponding to positions S184, N206, and V210 of SEQ ID NO: 2.

18. The method of claim 17, wherein the variant protein comprises the following amino acid substitutions:
S184H and N206M;
S184H, N206M, and T208Q; or
S184H, N206M, and V210D.

19. The method of claim 16, wherein the recombinant microorganism belongs to the genus Escherichia, Pseudomonas, or Bacillus.

20. The method of claim 16, wherein the contacting comprises culturing the recombinant microorganism with the sample comprising the fluorine-containing compound.
